(19) United States
(12) Patent Application Publication
(10) Pub. No.: US 2003/0195435 A1
(43) Pub. Date: Oct. 16, 2003

(54) Method and apparatus for collecting and transporting capillary blood samples for diagnostic and research evaluation

(76) Inventor: Arthur G. Williams, Thomasville, GA (US)

Correspondence Address: Ralph A. Dowell, Dowell & Dowell, P.C., Suite 309, 1215 Jefferson Davis Hwy, Arlington, VA, 22202, US

(21) Appl. No.: 10/120,483

(22) Filed: Apr. 12, 2002

(51) Int. Cl. A61B 5/00

(52) U.S. Cl. 600/583

(57) ABSTRACT

A method and collection and transport device for collecting biological samples, such as a blood sample, from a subject wherein the method and device include a body having a slide member mounted therein and movable from a first position wherein a collection well of the slide member is aligned to receive a sample from the subject to a second position wherein the sample communicates with a chemical reagent contained within a cavity of the body. The sample well and chemical reagent cavity are sealed from the exterior of the body when the slide member is in the second position. In preferred embodiments, locking means are provided to retain the slide member in the second position. In some embodiments, a lancet mechanism may be provided with the transport and collection device.

### BACKGROUND OF THE INVENTION

1. Field of the Invention

The present invention relates to methods, devices and kits for collecting and transporting capillary blood samples taken from human and animal subjects to designated testing laboratories or other facilities for the purpose of performing diagnostic and/or research evaluation procedures or tests of target capillary blood constituents typically known to be of clinical or diagnostic significance in the diagnosis, treatment and/or monitoring of specific diseases, states and/or chronic conditions.

In the present invention the subject clinical condition might be diabetes mellitus, all forms, and the target analyte (blood constituent) is A_{1c} (glycohemoglobin or GHb, also known as Total Glycohemoglobin, Hemoglobin A₁ and Hemoglobin A_{1c}), used in the diagnoses, treatment and/or management of this chronic long-term condition.

Such capillary blood sample collection/transport methods and devices have proven useful in several clinical/diagnostic applications such as screening for, diagnosing, treating and/or the routine monitoring of subjects with various clinical/diagnostic conditions such as diabetes.

The capillary method of sample collection has proven extremely useful in clinical/diagnostic practice by allowing and promoting routine clinical chemistry tests to be performed using small micro-samples of blood (1 μl to >100 μl) in place of much larger volumes (1 ml to 100 ml vacutainer tube) typically associated with veni-puncture sample collection procedures. Of particular significance is the use of capillary sample collection in pediatric and difficult to draw (overweight/collapsed vein/needle phobic) patients which would otherwise require a painful and often emotionally disturbing veni-puncture, arm vein draw collection procedure.

Capillary blood sampling methods and devices enhance the opportunity for improved incidence and frequency of routine monitoring of important at-risk indicators and/or compliance markers in various disease states. By monitoring these markers or indicators on a routine basis, the patient's chances for improved clinical outcomes and/or early diagnosis can be improved.

By utilizing capillary sample collection methods, devices and kits more effectively, a patient and a health care professional responsible for the care of the patient, are provided a convenient, non-traumatic, cost effective, reliable and easy to use method to routinely monitor specific outcomes markers and indicators, thus improving chances for appropriate clinical intervention and implementation of clinical treatment strategies.

2. Description of the Related Art

Capillary sample collection methods, devices and kits are currently used in a wide-variety of clinical or diagnostic applications. One such application involves the monitoring of mean blood glucose (MBG) in individuals with all forms of diabetes as derived from the direct determination (measurement) of HbA_{1c} or hemoglobin A_{1c}, a normal constituent of human blood. The normal range of this analyte in individuals that do not have diabetes is 4.3 to 5.5%. Mean blood glucose, is monitored as a measure of the relative level of management and control of the glycemic status of an individual patient over 90 to 120 days (the life of the circulating erythrocyte or RBC). In the case of diabetes, prevalence studies indicate that over 8 million individuals in the U.S. have some 20 forms of diabetes with another 16 million undiagnosed. In the case of hemoglobin A_{1c}, this represents a potential pool of over 32 million tests/year based on the American Diabetes Association “Standards of Medical Care” recommending each person be monitored at least four (4) times per year.

Currently, the incidence of individuals receiving this important routine test is less than 29% of the population of individuals with diabetes, representing an enormous opportunity for improved health outcomes in individuals with diabetes.

The reason for this low incidence of routine HbA_{1c} testing is several fold including the lack of awareness of the importance of the test by the general population and of general practitioners treating these patients, as well as the lack of a reliable, convenient, cost effective, method to collect and transport patient samples without fear of interference's, literacy level of patients, degradation of the sample stability by temperature and improper handling of samples by individuals collecting the samples. This test is considered vital to the long-term well being of individuals with all forms of diabetes. HbA_{1c} has been recognized by the NIH/NIDDK/ADA/AACE/CAP and the DCCT as a direct method to assess the risk associated with possible long-term complication typically associated with elevated and sustained mean blood glucose levels (MBG) determined by % HbA_{1c}.

Currently the analytical method (high performance liquid chromatography) or HPLC has been determined to be the reference method for this analysis. The HPLC method has two possible methods for sample collection, traditional veni-puncture and capillary collection.

The two methods correlate to one-another (0.9978) perfectly providing comparable results and clinical interpretation. By designing and using a convenient capillary sample collection and transport method and device, an increasing number of people with diabetes, in a variety of locations such as clinics, physicians offices, nursing homes, health agencies, and even in-home, can gain excess to the technology without the need for more invasive inconvenient procedures such as veni-puncture, while receiving the highest quality results and improved, convenience and economics.

The HPLC method is consider the most accurate, precise and reliable procedure for the determination of HbA_{1c} by the College of American Pathologists and the National Institutes of Health (published guidelines for the determination of hemoglobin A_{1c} “New England Journal of Medicine”). An historic landmark eight year study “Diabetes Control & Complications Trial” (DCCT) established that certain chronic complications associated with IDDM (Type 1 diabetes) could be prevented and/or delayed by as much as 72% by maintaining MBG at or about the upper limit of normal as measured by HbA_{1c} assay. Given this important landmark discovery, the need to establish an effective means to routinely measure HbA_{1c} in the general population of individuals with diabetes is paramount.

Currently, there are over 13 different analytical methods to measure various aspects of A_{1c} (glycohemoglobin). Only a few provide actual measurement of the A_{1c} fraction, the clinically significant and recognized indicator of mean blood glucose. Most methods read a total glycated hemoglobin, hemoglobin A_{1c}, and none have a capillary sample collection method to measure HbA_{1c} specifically without visiting an office or laboratory. The HPLC procedure is the only method today that can be collected in alternate sites other than a physician's office, laboratory or traditional medical facility. The complications associated with elevated and sustained levels of HbA_{1c} are well known and include, blindness, strokes, coma, end stage renal disease, micro/macro-vascular disease, amputations and death.

A_{1c} (glycohemoglobin) is the proteinaceous substance in the blood responsible for the proper transport of oxygen in the body. Current capillary sample collection procedures either employ a non-reference method blotter technology (filter paper technique) or a glass/plastic capillary tube ranging in size from 5 to 200 μl(s). The reference technology (HPLC) requires the use of an aqueous solution of EDTA and KCN to properly lyse (fix) the red blood cells and prevent further glycation of glucose once collected, while allowing for the detection of over 400 possible variant hemoglobin's that could be present in the sample.

Liquid transport/lysing media such as (EDTA) provides tremendous technical advantages over the filter paper (dried blood spot) technology. Currently, the EDTA/KCN liquid is contained in a 1.5 ml microfuge tube not unlike the traditional sample collection method of many other clinical/diagnostic procedures. In many cases, the technology uses a glass capillary tube, coated with sodium heparin, to collect the patient's blood sample from the finger which is than transported to a vial containing the specific pre-treatment and or fixing/lysing solution. Historically this has required the assistance of a highly trained phlebotomist and or trained health care professional, technical/medical assistant or worker.

Once the sample is collected it is typically transferred by being either dropped into the solution or the blood is expelled from the capillary tube directly into the solution for proper mixing and ultimate transport to a laboratory for analysis. This is a highly utilized practice in monitoring diabetes especially in the current reference (HPLC) method of sample collection and analysis. Currently, samples are then labeled and transported for analysis and ultimate patient results reporting. Blotter (filter paper) technology while in common practice in some tests, is not technically accepted and widely used in hemoglobin A_{1c} testing. Filter paper requires from 30 minutes to 24-hour specimen drying, measurement after a number of days (7-9), does not detect variant hemoglobins, has humidity problems, factors an HbA_{1c} and has a long history of unacceptable intra/inter laboratory quality control performance.

The current HPLC capillary collection (wet) method uses a 1.5 ml vial of EDTA with removable cap, a 5 μl glass Na heparinized capillary tube, and a plastic capillary holder (modified electronic clip). During the past three years, the inventor, in collaboration with major medical schools, conducted clinical trials to demonstrate the successful use of the capillary collection procedure in specific and limited populations of individuals with diabetes at alternate sites or using home collection procedures. During this time, the inventor and the clinical centers responsible for the trials have noted the acceptance and success of the procedures in the limited population.

In order for wide-spread utilization and adaptation of the A_{1c} procedure by the general population of diabetes patients, the need exists for a simplified procedure to collect the samples and transport them to a diagnostic reference laboratory or medical center for analysis and results reporting.

The current HPLC capillary sample collection procedure requires the following steps: a 1.5 ml vial containing EDTA is placed (held) in an upright position; the vial cap is opened; the patient pricks a finger to draw a small drop of blood; the patient then attaches the glass capillary tube to the capillary holder; one end of the capillary tube is then inserted into the drop of blood; the blood migrates up and fills the tube; the tube is removed from the sample and placed (dropped) into the EDTA solution; the cap is closed; the sample is mixed by rotation for a few seconds (10-15) until the blood in the capillary tube is completely removed (diluted); the patient ID label is placed on the vial; and the vial is placed in a protective mailer and sent to the laboratory for analysis. The patient is typically supplied with a kit that contains supplies and materials for several samples. The alternate site sample collection kit is intended for unqualified (OTC) use. The same limitations that apply to blotter technology also apply in that the individual must first desire to collect their sample in this fashion, be able to visually and mentally complete the task of sample collection and be literate enough to follow directions. A desire of the trial coordinators, individuals participating in the trials and the commercial industry has been to remove the glass capillary tube and the possible exposure to the lysing solution (EDTA) without compromising current standards of quality and accuracy of the procedure achieved by HPLC.

To date, there is no known self-contained (closed) aqueous capillary sample collection and transport method or device to collect individual samples that did not compromise the precision and accuracy of the specific results such as A_{1c} (especially dried blood spot procedures). Thus, there is a need in the art to provide a more convenient, simplified method and device to collect, prepare and transport capillary blood samples while utilizing existing sample volumes, proven clinical protocols, comparative clinical correlation data (DCCT), commercially available solutions in known concentrations, such as EDTA and KCN, proven QC materials and automated analytical instrumentation, without the concern of possible exposure by the patients and health care professionals to such substances along with the removal of the manipulation attachment steps for securing, transporting and using glass/plastic capillary tubes.

### SUMMARY OF THE INVENTION

In accordance with the present invention, a method and device for lancing, receiving, mixing and transporting a finger-stick (capillary) blood sample from a subject includes the subject collecting a small volume (drop) of the subjects own whole blood from a finger using a self-embodied lancet associated with a collection and transport device. The subject or care provider applies the drop, approximately 5 μl's of the freshly collected blood, to a designated sample collection compartment or sample well positioned atop a main body of the collection/transport device. The subject is instructed to remove their finger from the top of the device leaving the residual blood behind in the designated sample collection well of determined volume located in a movable component or slide of the collection device. The subject/care provider is instructed to move the one-way, self-locking sample transport slide from an open position containing the collected sample to a closed (mixing) position located in close proximity (approximately <⅛ of an inch away) indicated by the alignment of a universally recognized + (PLUS) sign in the sample well location. When in this position, the collected blood sample is exposed, for the first and final time, to an aqueous solution of EDTA/KCN (or designated chemical (s)) through a specified diameter opening located and aligned with a reagent well containing the designated reagents(s). The fresh blood sample is now positioned directly above the reagent well or a membrane sack and due to gravity and diffusion principles will automatically mix and create a diluted preparation of blood and chemicals. This principle is possible due to the fact that the sample well is open at the bottom and top. The collection/transport device may also include features such as a positive ID bar-code label, affixed on the body of the device.

A kit used with the device may further include an optional carbonless (two-part) laboratory ID form. The subject is instructed to locate this form from the kit, remove, complete and retain the back copy for their records and reference. The subject is further instructed to place the device containing their blood sample in a bio-hazard specimen bag (Bio-Pack) which is provided as a component of the sample collection kit. The subject is instructed to seal the Bio-Pack by removing and pressing a self-adhesive tape attached to the top of the Bio-Pack. The subject is further instructed to place the Bio-Pack containing the collected/mixed sample in a pre-addressed-postage paid mailer provide in the kit, seal it and place it in the mail for transport (mailing) to a designated laboratory for analysis and results reporting.

Also in accordance with the present invention, the sample collected within the device may be sampled (analyzed) via a plug located in direct proximity of the reagent well and constructed of a suitable substance that allows penetration of the plug via a probe designed to withdraw a specified volume of the patient sample mixed in the designated reagent(s) for analysis of the desired analyte. The reagent well probe plug is preferably located at an end of the device.

### BRIEF DESCRIPTION OF THE DRAWINGS

A better understanding of the invention will be had with reference to the accompanying drawing figures wherein:

FIG. 1 is a perspective illustrated view of a collection and transport device in accordance with the invention;

FIG. 2 is a top plan view of the device of FIG. 1;

FIG. 3 is a cross-sectional view taken along line 3-3 of FIG. 2;

FIG. 4 is a cross-sectional view taken along line 4-4 of FIG. 2;

FIG. 5 is a cross-sectional view taken along line 5-5 of FIG. 2;

FIG. 6 is a cross-sectional view taken along line 6-6 of FIG. 2 illustrating the collection of a blood sample within a well of a slide member of the device;

FIG. 7 is a cross-sectional view similar to FIG. 6 except showing the slide member moved to a second locked position and illustrating the mixing of the blood sample into chemical reagent(s) and the withdrawal of a test sample from the device using a probe;

FIG. 8 is an enlarged partial cross-sectional view showing a slide member locking device;

FIG. 9 is a bottom plan view of the device of FIG. 1; and

FIG. 10 is a top plan view of a bio-pack in which the device of FIG. 1 is protectively sealed for mailing or transport.

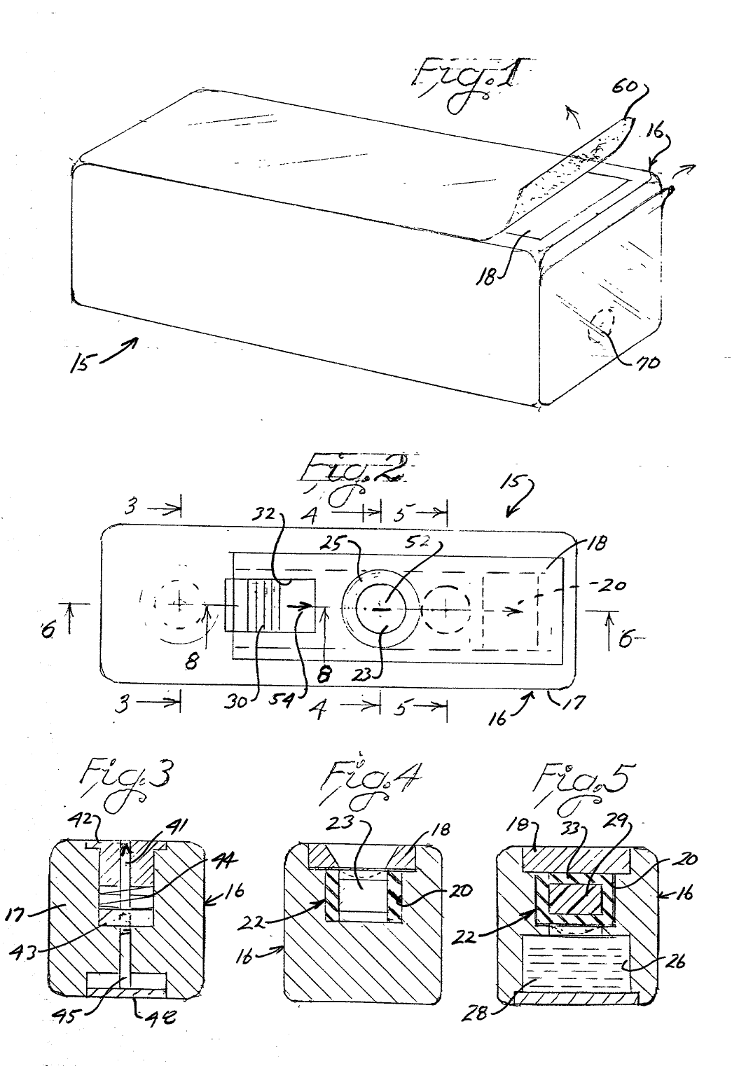

### DESCRIPTION OF THE PREFERRED EMBODIMENTS

According to the present invention, a method for lancing the finger, collecting a blood drop, preparing or mixing the blood and chemicals and transporting a capillary blood sample from a subject to a designated testing laboratory is provided. The method of the present invention includes the subject lancing a finger using a self-embodied lancet or subjects own lancet, and drawing a small amount of the subjects own capillary blood. The subject applies a drop of capillary blood to a designated opening or a sample application well located and depressed in a top of a collection and transport device. The subject or care provider then moves a one-way, self-locking sample transport slide from an open collection position to a closed and locked mixing position located a small distance away and in alignment with an opening into a reagent well. The sample and chemical(s) within the reagent well are automatically mixed in the reagent well. The subject may thereafter complete and retain an optional copy of a lab ID form. The subject places the device in a Bio-Pack (OSHA/USPS approved specimen transport bag) for transport of the specimen to a designated laboratory. The Bio-Pack is placed in a designated sample mailer which is mailed to a laboratory for analysis and results reporting.

The designated laboratory receiving the device places the device in a position on an automated or manual analytical instrument, such as an HPLC system, to withdraw a specified sample volume of the patients sample via mechanical means, such as a probe.

Further according to the present invention, a collection and transport device 15 for the collection, mixing and transport of a capillary sample of blood “B” from a subject is provided. The device includes a molded housing 16 constructed of polypropylene or equivalent plastic. The housing component is constructed of a hard plastic body 17 having a sealed cover 18 which defines a channel or slot 20 in which a slide 22 containing a specified volume sample application well 23 is movably mounted. The well 23 is used for receipt of a drop of whole blood or human biological specimen or sample which is received through a funnel-shaped opening 25 in the cover 18. The housing also has a sealed, leak proof, mixing cavity or compartment 26 for the storage of designated reagent(s) or solution(s) 28. The volume of reagent(s) will vary depending on various diagnostic analytical applications using whole blood or human biological specimen, such as EDTA. The slide is fitted within the slot so as to serve as a self-sealing member so that the reagent(s) or solutions within the cavity 26 can not leak. In this respect, after a sample has been collected and mixed within the device, the mixed sample is safely contained and will not pose a danger to individuals handling the device.

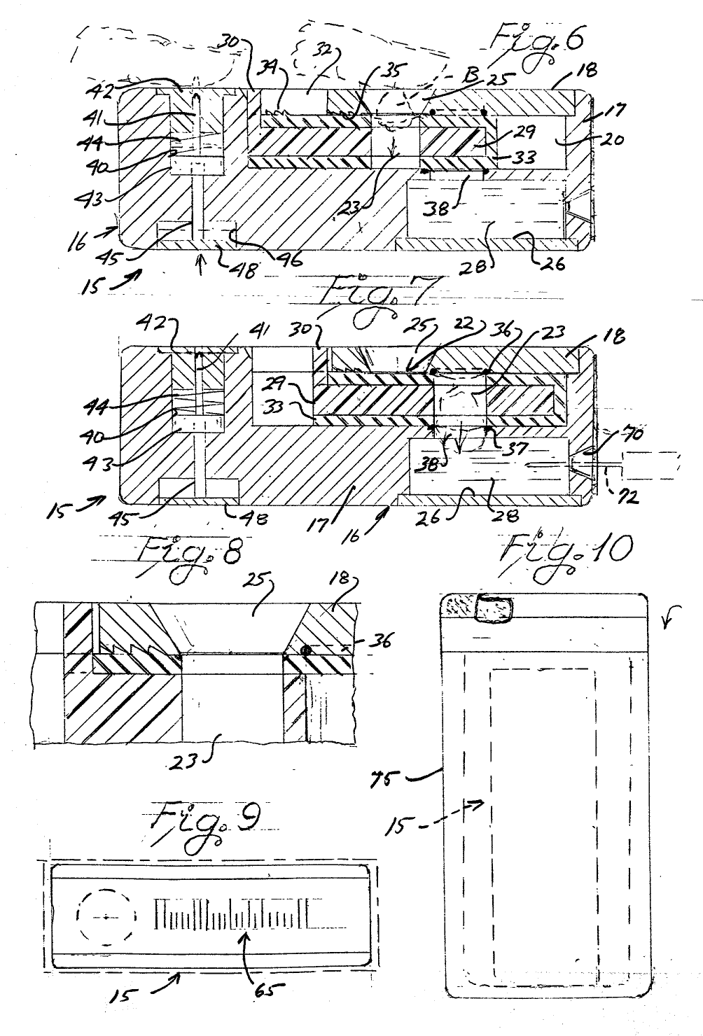

As shown, the slide 22 may be formed of a central body 29 molded of a hard plastic similar to that used to mold the housing 16. The slide body includes an integral push tab 30 which extends upwardly through an opening 32 provided between the body 17 and cover 18 of the housing 16. In this manner, the tab is engageable by a person's finger or thumb so that by appropriate application of force, the slide may be moved from a first collection position, as shown in FIG. 6, to a second and locked mixing and transport position, as shown in FIG. 7. As opposed to extending the tab 30 through an opening in the top of the device, the tab could be extended through an opening in a side or bottom wall of the housing.

Although the slide may be formed as a single integral component of a size to be frictionally and yet, slideably, received within the channel 20, as shown, in order to provide for a continuous seal between the slide and channel, the central body 29 may be enclosed within a jacket 33 formed of a natural or synthetic rubber or like material which will provide a leak proof fluid seal between the slide and housing regardless of the position of the slide.

To prevent any movement of the slide after it is has been moved to the mixing position shown in FIG. 7, a locking arrangement is provided between the slide and housing. In the embodiment shown, the slide may include a plurality of locking ratchet-like teeth 34 which are slightly resiliently yieldable to permit movement of the slide from the first to second positions but which will intermesh and lock with opposing teeth 35 proved in the base of the cover 18. Other types of positive locking members may be used.

To further provide a leak-proof seal to prevent any lost of fluid from the mixing cavity 26, appropriate annular o-rings 36 and 37 may be seated within annular recesses provided in the base of the cover and a wall defining a mixing opening 38 between the collection well 23 and cavity 26. In this respect, it should be noted that the collection well is open at the top and bottom and is positioned to be aligned with the opening 25 in the first position and with the mixing cavity when the slide is moved to the second, mixing position.

The precise volumetric capacity of the sample or collecting well 23 is measured in μl's and is accomplished or defined via either the defined thickness of the slide 22 or the diameter of the well.

The device 15 may also include a cavity or compartment 40 containing a lancet device consisting of a calibrated stainless steel (sharp) lancet 41 or equivalent device. The lancet is normally covered by and partially housed within a protection cap 42 and a base 43 of the lancet in a normally urged by a spring 44 within the cavity 40. The lancet base 43 is also engaged by an activator mechanism 45 mounted in a cavity 46 aligned with cavity 40. The activation mechanism includes a push button 48 which is manually activated to urge the lancet base against the spring 44 to cause the tip of the lancet to penetrate the cap 42 to pierce the skin of an individual's finger, as illustrated in dotted lines in FIG. 6. Upon release of the button 48, the spring 44 will withdraw the lancet into a safe and non-hazardous position within the cavity 40.

After the finger has been pierced, a drop of blood “B” is deposited through the opening 25 and into the sample collection well 23, also as shown in dotted line in FIG. 6.

As noted, according to the present invention, the sample slide is movable in only one direction and is prevented from backward movement by the one-way lock mechanism when the slide is moved to the permanent second position or mixing position. An indicator (not shown) is visible as a PLUS (+) sign through the opening 25 in the cover of the housing when the slide is moved to the second mixing position. To facilitate proper use, the direction of movement of the slide is directed by an embossed arrow 54 on the top of the sample slide and visible to the subject for confirmation of position prior to beginning the mixing step. Also, a marking 55, such as a minus sign “−” may be visible through opening 25 prior to the device being used to thereby indicate that the device is ready for use to collect and mix a sample and the slide is in the first position.

Further according to the present invention, the opening 25 in the cover 18 into the sample well is preferably tapered (a funnel shaped opening) and defines a depression approximately the size of a adult male index finger pad. The blood sample to be contained is directed and funneled down and into the sample well 23.

According to the preferred embodiment of the present invention, the opening 25 is aligned in direct proximity to the sample well 23 in the first position so that the required or defined volumetric capacity of the subject's blood sample, measured in μl's, is collected by direct access to the sample well from the opening.

Further according to the present invention, the opening 25 to the sample well is protected by a tamper indicating seal 60 that must be removed by the subject prior to collecting the capillary blood sample. The tamper indicating seal includes an adhesive substance that affixes to and over the housing cover. As an alternative, the device may be contained within a sealed pack, to provide evidence of tampering or unintentional discharge of the lancet device. Also, in preferred embodiments, to insure accuracy, the housing 16, as shown in FIG. 9 which is coded as necessary to provide patient and/or other date and information.

As previously described, the sample transport slide is constructed of either a plastic or hard rubber-like (gasket) material that contains a pre-defined and measured opening for the sample well that is calibrated to a specific volumetric capacity measured in microliters (μl's). Furthermore, the sample well is contained within the channel of the housing in tight proximity to all sides of the channel thereby preventing leakage during sample collection, storage and transport to a testing laboratory and thus preventing exposure of the aqueous reagent solution(s) to the subject. Furthermore, the sample well of the slide is designed in a variety of volumetric capacities measured in μl's ranging from 1 to approximately >100 μl's, in accordance with the diagnostic application requirement.

The collection and transport device, after being received at a testing laboratory, is placed in an upright position on a sample rack such that the mixing or reagent cavity 26 is accessible through a probe plug 70 which normally seals the cavity. The plug is penetrable by a probe 72 through which the mixed sample/reagent is withdrawn from the cavity. As an example: a manual or automated analyzer such as a HPLC system (Primus SC335 HPLC system) for specific analysis of the desired diagnostic test. Preferably, the device is contained within an OSHA/Postal Service approved specimen transport bag/container (not shown) for shipment (transport) to the designated laboratory. Preferably, the collection and transport device is inserted and sealed in a container 75, which prevents breakage, spillage or contamination of the device during transport.

In an especially preferred embodiment of the present invention, a patient draws his or her own capillary blood sample at a non-traditional location, such as the home, thus avoiding the need to visit a medical facility or laboratory drawing station. The patient safely and conveniently collects their capillary blood sample in the privacy of their home or other alternate site sample collection location, using the device for such purposes, and mails it to a medical laboratory for analysis. The invention thus provides the patient with the convenience and safety of home or alternate site collection, FDA approved collection devices/methods, while providing the technical advantages of precision, accuracy and analytical excellence, available only by aqueous methods of sample analysis (or reference methods such as HPLC), while simultaneously avoiding the time, inconvenience and expense of traditional sample collection procedures in medical facilities.

The present invention recognizes the opinion of the clinical chemistry community surrounding the analytical excellence that can only be derived by the use of certain aqueous reagent solutions such as EDTA in the liquid state to detect such important variants as hemoglobiopathies such as (sickle cell anemia) in individuals being monitored for A_{1c}, and recognizes that this aqueous solution(s) and/or reagent(s) method of analysis is the only method that allows detection of such hemoglobinopathies including the use of filter paper (blood spot) techniques and methods. The present device and method provides the patient and medical community the ability to realize analytical excellence and technical efficacy in performing certain diagnostic procedures such as A_{1c}. With the diagnostic knowledge that this type of testing provides, the over all diagnostics of the patient can be enhanced and ultimately provide improved medical care.

According to another preferred embodiment of the present invention, a kit for collecting a capillary sample in a subject is provided. The kit of the present invention includes the shipping/storage container 75 made of a suitable material which permits the holding, storing and transporting of the individual kit components without subjecting the components to harm or, contamination. Preferably the container is made of either plastic, corrugated cardboard and/or sealed aluminum foil. Moreover, the container can optionally possess (hold/store) at least one, but preferably more than one capillary collection and transport device with related supplies for collection/transport of a specific number of samples.

The capillary sample collection kit includes at least one capillary blood sample collection and transport device, at least one OSHA approved bio-pack specimen bag 75 designed for transporting the sample; at least one chipboard or suitable material mailer (box/bag/envelope), not shown, to house the device containing the sample, at least one patient multi-part laboratory identification ID form with number or bar code; and at least one lancet device if not included with the transport and collection device itself, as in the preferred embodiment.

The kit optionally can include other items such as sample collection instructions (printed/schematic illustrations), information, resource materials, protocols for clinical trials/studies, general disease/subject matter materials, diagnostic tracking logs (graphics representing ranges of specific analytes measured), pre-addressed mailers, phone numbers for technical support additional information.

While the invention has been described and illustrated with details and references to certain preferred embodiments, those skilled in the art will appreciate that various modifications, changes omissions and substitutions can be made without departing from the spirit of the invention.

The new method and device allows for wide-spread use of remote/alternate site sample collection, promoting increased use of specific diagnostic testing/monitoring due to improved patient and health care system convenience and economics.

The present invention provides a safe, convenient and accurate method for allowing the patient and health care professional (provider) to collect capillary samples from patients, without the customary requirement and costly assistance of a health care professional and/or laboratory personnel, thus promoting increased frequency and compliance of performing specific diagnostic tests for initial and on-going monitoring of chronic diseases, such as diabetes.

What is claimed is:

1. A method for collecting and transporting capillary blood and/or biological samples using a collection and transport device having a slide mounted therein and wherein the slide includes a sample receiving well which is movable from a first collecting position for receiving a sample to a second position in which the sample may be subjected to a chemical reagent contained within the device, the method comprising the steps of:
a. obtaining a small capillary blood or biological sample and placing the sample the collection well;
b. moving the collection well from the first position to the second position wherein the collection well is aligned such that the sample contained therein is subjected to the reagent within the device and such that the reagent and the sample are completely sealed within the device;
c. conveying the device to a testing facility;
d. at the testing facility withdrawing the sample subjected to the chemical reagent from the device so that the sample may be analyzed; and
e. thereafter analyzing the sample and reporting the results of the analysis.

2. The method of claim 1 including the additional step of applying a scanable code to the collection and transport device including identifying data with respect to the subject prior to the subject obtaining the sample and placing the sample within the collection well.

3. The method of claim 2 including the additional step of providing an approved biological mailer, placing the device with the sample and chemical reagent within the approved device prior to conveying the mailer to the testing facility.

4. The method of claim 3 including the additional step of providing a tamper indicating seal over the collection well of the device prior to supplying the device to the subject, and removing the tamper indicating seal prior to placing a blood sample within the collection well.

5. The method of claim 1 wherein the sample is capillary blood and the capillary blood and chemical reagent are mixed within the device.

6. An apparatus for collecting and transporting a biological sample such as a capillary blood sample, from a subject such that a sample may be taken in a first location, placed in a device and the device transported to a second location wherein the sample is tested and analyzed, the device including; a body having a channel formed therein spaced from an upper surface thereof, a first opening in said upper surface communicating with a first portion of said channel, a slide member mounted within said channel and being movable with respect thereto, means for sealing said slide member in fluid tight relationship with respect to said channel, a reagent cavity formed within said body and having a chemical reagent contained therein, means for communicating said cavity with said channel, a collection well formed within said slide member for receiving a sample from a subject when the slide member is in a first position wherein said well is aligned with said first opening in said upper surface of said body, said slide member being movable to a second position wherein said collection well is aligned such that a sample contained therein may communicate with the reagent contained within said cavity, and means for sealing said cavity but allowing a probe insertion into said cavity through said body such that a sample reacted with the chemical reagent may be withdrawn therefrom.

7. The apparatus of claim 6 in which said collection well defines an opening through said slide member and wherein the dimensions defining said opening through said slide member may be varied to effectively control an amount of sample contained within the said collection well.

8. The apparatus of claim 7 in which said slide member includes an operating tab extending outwardly of said body so as to be engageable to urge said slide member from said first to said second position.

9. The apparatus of claim 8 including a second opening in said body, said tab extending through said second opening and said second opening being spaced from said first opening.

10. The apparatus of claim 8 including a lancet means mounted within said body, and means for activating said lancet so as to extend outwardly of said body and thereafter be withdrawn within said body.

11. The apparatus of claim 10 in which said lancet means includes a resilient element for normally urging said lance means within said body.

12. The apparatus of claim 11 in which said lancet means is normally covered by a protective seal extending across a surface of said body.

13. The apparatus of claim 7 in which said first opening in said body includes an inwardly tapering side wall forming a funnel which is aligned with said collection well of said slide member when said slide member is in said first position.

14. The apparatus of claim 7 including indicia means associated with said slide member for indicating said slide member is in said first position and second indicia means associated with said slide member for indicating that said slide member is in said second position.

15. The apparatus of claim 7 including locking means for locking said slide member in said second position when moved to said second position from said first position.

16. The apparatus of claim 15 in which said locking means includes elements for preventing said slide member from being moved from said second position to said first position.

17. The apparatus of claim 7 including scanable code means provided along an outer surface of said body member for providing information with respect to the subject.

18. The apparatus of claim 7 including a bio-protective mailer of a size to receive said transport and collection device therein.
